(19) United States
(12) Patent Application Publication
(10) Pub. No.: US 2026/0251169 A1
(43) Pub. Date: Aug. 27, 2026

(54) Fastening Part

(71) Applicant: A. RAYMOND ET CIE, GRENOBLE (FR)

(72) Inventors: Romain MOUILLON, VOREPPE (FR); Jean-Francois ALBARAN, Varces Allières et Risset (FR); Anthony OULES, GRENOBLE (FR)

(21) Appl. No.: 18/877,041

(22) PCT Filed: Jul. 10, 2023
(86) PCT No.: PCT/EP2023/069062

§ 371 Date: Dec. 19, 2024

(30) Foreign Application Priority Data
Jul. 8, 2022 (FR) 2207055

(51) Int. Cl. F16B 13/02; F16B 5/06

(52) U.S. Cl. CPC F16B 13/02; F16B 5/0642; F16B 2200/95

(57) ABSTRACT

A fastening part, for fastening a first part on a second part having a hole, includes a body which includes: a head having a rear surface intended to be positioned opposite the first part, and a shank extending from the rear surface of the head and intended to be inserted into the hole. The shank includes fins which project from the shank, and at least one fin includes, at its free end, a double-head serving as a thread.

### CROSS-REFERENCE TO RELATED APPLICATIONS

This application n is a National Stage of International Application No. PCT/EP2023/069062, having an International filing date of 10 Jul. 2023, which designated the United States of America, and which International Application was published under PCT Article 21 (2) as WO Publication No. 2024/008974, which claims priority from and the benefit of French Patent Application No. 2207055 filed on 8 Jul. 2022, the disclosures of which are incorporated herein by reference in their entireties.

### BACKGROUND

### 1. Field

The present disclosure relates to a fastening part, in particular a rivet intended to be inserted and mounted in a hole to fasten a first part to a second part, so-called the support. For example, the fastening part allows assembling hard materials (metal, steel, etc.) on hard materials for the automotive industry (installation of accessories, bodywork parts, etc.). More particularly, the rivet includes a shank having double-headed fins.

### 2. Brief Description of Related Developments

A rivet in the form of a shank which is provided at one of its ends with a head is known from the prior art. By “head”, it should be understood a portion with a larger section than the section of the shank.

The head of this kind of fastening part has a rear surface intended to be positioned opposite a part to be fastened. Preferably, the head also includes a front surface opposite to the rear surface.

The shank extends from the rear surface of the head and intended to be inserted into a fastening hole which is located in parts to be assembled by the fastening part. The head may be round or rectangular shaped. The cross-section of the shank may be circular.

This fastening part could be used, for example, to fasten a part on a structure or a support. In order to properly fasten this fastening part of the prior art, it is essential for the fastening part to be pushed deep enough into the fastening hole. Depending on the force exerted when pushing the fastening part, it is possible, for example, that the rear surface portion of the head of the fastening part is not in contact with the part to be fastened, thus the fastening part is not properly mounted, and it turns out to be complicated for the operator to verify the fastening state. In this case, the fastening part might come out during use of the parts to be assembled.

Progresses continue to be made in the improvement of a hooking system that addresses various needs such as rigidity, robustness of fastening (efficient ratio between the insertion force and the pull-off force), and still others.

### SUMMARY

The present disclosure aims to overcome these drawbacks, by providing a fastening part which allows having a better clipping blade end rigidity, a double hooking in the thread and a better unscrewing ability.

To this end, the present disclosure relates to a fastening part for fastening a first part to a second part having a fastening hole, including a body which includes:
a head having a rear surface intended to be positioned opposite the first part, and
a shank extending from the rear surface of the head and intended to be inserted into the fastening hole;

The shank includes hooking fins which project from the shank, and that at least one hooking fin includes, at its free end, a double-head serving as a thread.

In one aspect, said at least one hooking fin includes a thin area between the shank and the double-head.

In one aspect, the double-head includes an upper head and a lower head separated by an intermediate space, these two heads defining a thread pitch.

In one aspect, the fins are fir tree like shaped.

The flexible tab comprises an elastic arm, one end of which is secured to the body of the fastening part. The arm includes a control.

When the fastening part is installed in the part(s) to be fastened, the arm of the flexible tab deforms so that the control moves to fit in a predefined position with respect to the head of the fastening part.

With the movement of the control, the operator visually sees that the fastening part is properly mounted.

Advantageously, the control moves towards the head of the fastening part in the mounting position. Normally, the head of the fastening part and its surrounding are clearly visible, thus the operator verifies more clearly whether the control properly fits in the predefined position.

Preferably, the shank of the fastening part includes fins projecting from the shank, serving as a thread which prevents the fastening part from coming out of the fastening hole, thereby making fastening more solid.

Preferably, the head includes at least one notch intended to receive the control in the mounting position of the fastening part. The notch serves as the predefined position where the control fits in. This allows for a more clearly visible indication for the operator, especially when viewed from above. Once the control fits in the notch, the operator visually sees that the fastening part is properly mounted.

In one aspect, the head of the fastening part includes at least one setback, intended to accommodate the control when the control fits in the predefined position, and to prevent the control from moving during unscrewing. This configuration allows the head to be flush during unscrewing, for reasons related to tooling.

Preferably, the setback is in the predefined position which receives the control in the mounting position. Advantageously, each predefined position includes two setbacks for accommodating the control in a more balanced manner.

Advantageously, in the mounting position of the fastening part, a surface of the control coincides with a front surface of the head, so that the two surfaces are flush. This means that there is no step between the surface of the control and the front surface of the head. By front surface of the head, it should be understood the surface opposite to the rear surface of the head. In this aspect, if the fastening part is not properly mounted, the operator can know it by noticing an unevenly planar surface of the head when touching the front surface, especially when the light of the environment during the process of fastening using the fastening part according to the present disclosure does not enable the operator to see clearly.

In one aspect, the arm of the flexible tab is secured to the rear surface of the head to have a more compact structure of the fastening part.

Advantageously, the flexible tab is positioned on the side of the rear surface of the head, in a released position, i.e. when the fastening part is not mounted in the part(s) to be fastened and/or is not subjected to any stress. This arrangement enables the flexible tab not to prevent the process of mounting and dismounting the fastening part, thus the operator mounts and dismounts the fastening part according to the present disclosure like a conventional fastening part in the prior art, and no attention or special and/or additional effort is needed.

Advantageously, the arm of the flexible tab extends towards the edge of the head. Thus, the control, which can be connected to the free end of the arm, is close to the edge of the head. This facilitates the manufacture of the fastening part according to the present disclosure.

In one aspect, the fastening part includes at least one pair of flexible tabs. The two flexible tabs of the pair of flexible tabs extend in two opposite directions. Thus, the two controls of the two flexible tabs are located in two opposite sides of the head, which allows showing that the fastening part is properly mounted in a balanced manner when both controls fit in the predefined positions. Preferably, the two flexible tabs of the pair of flexible tabs are identical. This facilitates the manufacture of the fastening part. In the proper mounting position, the head of the fastening part is parallel to the surface of the part to be fastened which is in contact with the head, when the surface of the part is evenly planar.

In one aspect, the head shape is round, and the two flexible tabs of the pair of flexible tabs extend in two diametrically-opposite directions.

In one aspect, the head of the fastening part has an imprint for receiving a tool. Preferably, the tool received by the imprint is used to mount and/or dismount the fastening part. This facilitates mounting and/or dismounting of the fastening part.

Preferably, the imprint is located at the centre of the front surface of the head.

Advantageously, the fastening part is made of a plastic material.

Advantageously, the fastening part includes metal components, so-called claws, projecting from the shank and extending towards the head so that the fastening part is more solid in the fastening hole of the parts to be fastened.

### BRIEF DESCRIPTION OF THE FIGURES

Other advantages, aims and features of the present disclosure will become apparent from the following description made, for explanatory and non-limiting purposes, with reference to the appended drawings, wherein:

FIG. 1 shows a sectional view of the first part and the second part to be fastened by a fastening part according to one aspect, in which each of the two parts to be fastened has a fastening hole;

FIG. 2 shows a perspective view of a fastening part according to one aspect;

FIG. 3 shows another perspective view of a fastening part according to one aspect;

FIG. 4 shows a side view of a fastening part according to one aspect;

FIG. 5 shows a top view of a fastening part according to one aspect;

FIG. 6 shows a side view of a fastening part according to another aspect;

FIG. 7 shows a detail view of the area A in FIG. 6;

FIG. 8 shows a sectional view of a fastening part according to one aspect, the rivet being received in a threaded hole;

FIG. 9 shows a similar view of a fastening part according to one aspect, the rivet being received in a threaded hole;

FIG. 10 shows a detail view of the area B in FIG. 9;

FIG. 11 shows a sectional view of the double-headed portion of a fin according to one aspect.

### DETAILED DESCRIPTION

The different figures as well as the elements of the same figure are not necessarily plotted according to the same scale. In all figures, identical elements bear the same reference numeral.

The terminology used in the present description should in no way be interpreted in a limiting or restrictive manner, simply because it is used in conjunction with a detailed description of some aspects of the disclosure.

FIG. 1 shows a sectional view of the first part 200 and the second part 400 to be fastened by a fastening part 100 according to an aspect of the present disclosure. In FIG. 1, the fastening part 100 is still not mounted. Preferably, the first part 200 may have a first hole 220, or an opening of another type, for example, a slot (not shown in the figure). The second part 400 has a second hole 420. Just as example, the first part 200 may be an M6×100 type nut, and the second part 400 may be a sheet metal on which the M6×100 type nut is welded.

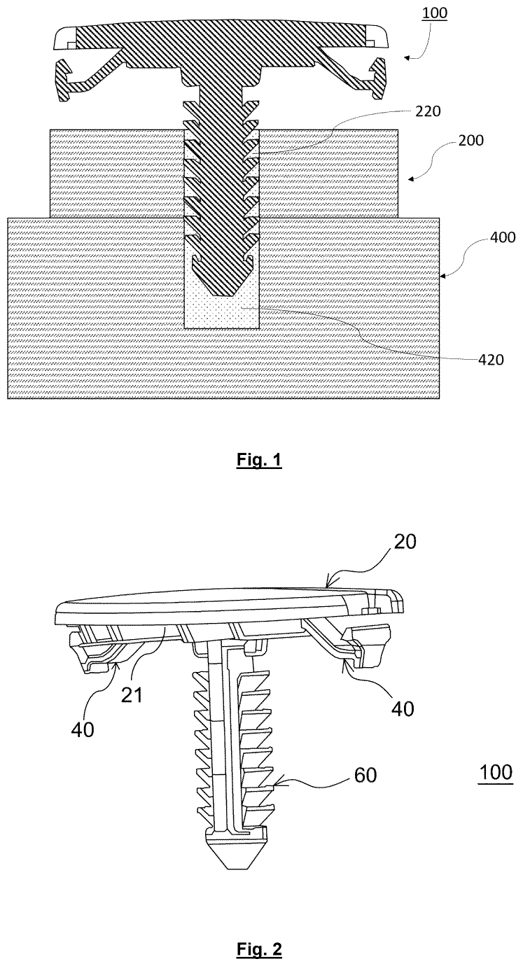

FIG. 2 shows a perspective view of a fastening part 100 according to an aspect of the present disclosure. The fastening part 100 includes a body which comprises a head 20 and a shank 60. The head 20 has a rear surface 21 intended to be positioned opposite the first part 200. The shank 60 extends from the rear surface 21 of the head 20 and is intended to be inserted into the hole(s) of the parts to be assembled.

Preferably, the head 20 is round shaped. Preferably, the shank 60 is cylindrical shaped.

The term “stem” used in this description may be technically substituted by the terms “trunk” or “central core”.

In one aspect (not shown), the fastening part 100 includes one single flexible tab 40. The flexible tab 40 deforms in a mounting position, in which the first part 200 is fastened to the second part 400 by the fastening part 100. The deformation of the flexible tab 40 enables the operator to visually see that the fastening part 100 is properly mounted. This part will be detailed later on.

In another aspect, the fastening part 100 includes a pair of flexible tabs 40, as illustrated in FIG. 2. Advantageously, the fastening part 100 includes multiple flexible tabs 40, which allows better indicating the proper mounting of the fastening part 100 when the configuration of the parts to be fastened is more complicated.

FIG. 3 shows another perspective view of a fastening part 100 according to an aspect of the present disclosure. The flexible tab 40 comprises an arm 44, preferably elastic, and the arm 44 includes a control 42.

When the fastening part 100 is properly mounted in the fastening hole of the parts to be assembled, i.e., when the fastening part 100 is in the mounting position, the arm 44 of the flexible tab 40 deforms so that the control 42 fits in a predefined position with respect to the head 20. Preferably, the predefined position is a notch 22 on the head 20, as illustrated in FIG. 3. The head 20 may have one or two or more notch(es). In FIG. 3, the head 20 comprises a pair of notches 22. Preferably, the number of notches 22 corresponds to the number of controls 42. Each notch is intended to receive one of the controls 42 in the mounting position of the fastening part 100.

In one aspect, the head 20 includes a setback 26. Preferably, the setback 26 is in the predefined position which receives the control 42 in the mounting position. Advantageously, each notch 22 includes two setbacks 26, as illustrated in FIG. 3. The setbacks 26 allow the unscrewing to be flush. These setbacks 26 are in opposition with respect to the control 42. The control 42 is accommodated against the setbacks. This shape is so for reasons relating to tooling. Fins 62 in FIG. 3 project from the shank 60.

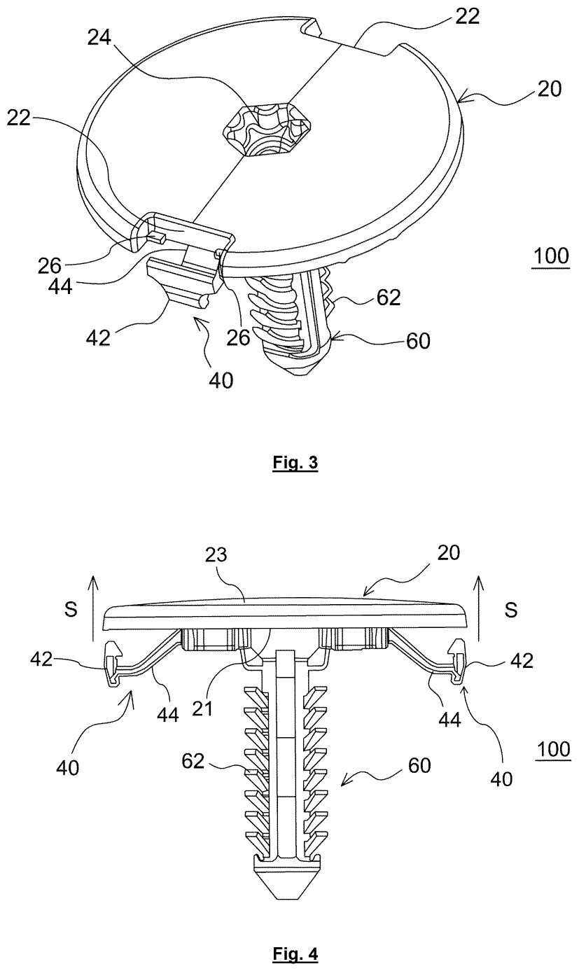

FIG. 4 shows a side view of a fastening part 100 according to one aspect. The fins 62 serve as a thread which prevents removal of the fastening part 100 and thus makes the fastening part more solid. Preferably, the fins 62 are in fir tree like shaped so as to guarantee an even more solid fastening. Just as example, the shank 60 with the fins 62 functions like the shank 60 of the conventional fir tree like shaped rivet. The fins 62 will block the fastening part 100 at the outlet. For example, the fastening part 100 with the fir tree like shaped fins 62 may be used to fasten a plastic deflector in an M6×100 threaded hole by clipping.

As illustrated in FIG. 4, one end of the arm 44 is secured to the body of the fastening part 100. Advantageously, the arm 44 is connected to the head 20 at the rear surface of the head 20. Preferably, the arm 44 extends towards the edge of the head 20. Thus, the structure of the fastening part 100 is more compact.

In one aspect, the flexible tab 40 is positioned on the side of the rear surface 21 of the head 20 in a released position, when the body of the fastening part 100 does not receive any external force, as illustrated in FIGS. 1, 2, 3 and 4.

Preferably, the control 42 is on the free end of the arm to have a compact configuration of the fastening part 100. Preferably, the control 42 is positioned on the side of the rear surface of the head 20, in the released position, as illustrated in FIG. 4.

In one aspect, the fastening part 100 includes a pair of flexible tabs 40. As illustrated in FIG. 4, the two flexible tabs 40 of the pair of flexible tabs extend in two opposite directions.

In one aspect, when the head 20 is round shaped, the two flexible tabs 40 extend in two diametrically-opposite directions. This makes the operator more assured that the fastening part 100 is properly mounted in a balanced manner.

Advantageously, the arm 44 and/or the control 42 comes into contact with at least one of the parts to be assembled in the mounting position and, preferably, the arm 44 and/or the control 42 deforms while coming into contact with at least one of the parts to be assembled, so that the control 42 rises in a direction S, as illustrated in FIG. 4. This means that the control 42 rises from the level of the rear surface 21 towards a front surface 23 of the head 20 which is opposite to the rear surface 21. The movement of the control 42 in the direction S is more visible than in other directions for the operator.

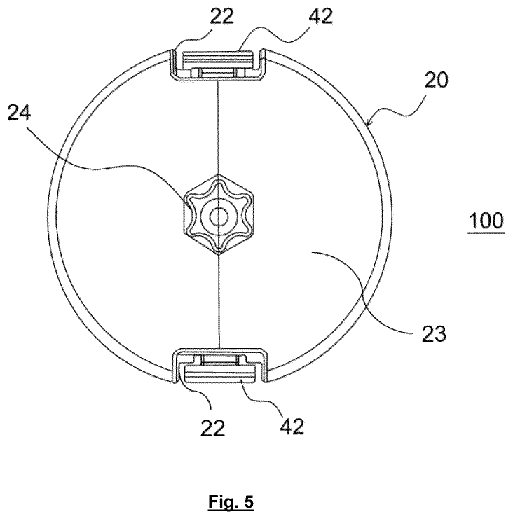

FIG. 5 illustrates a top view of a fastening part 100 according to an aspect of the present disclosure. Two notches 22 are located on the edge of the head 20. The head 20 is round shaped in this aspect, and the two notches 22 are diametrically opposed. The two notches 22, corresponding to the predefined positions in which each receives a control 42 amongst the controls 42 in the mounting position.

Advantageously, the control 42 moves until a surface of the control 42, for example, a surface of the control 42, coincides with the front surface 23 of the head 20 in the mounting position of the fastening part 100. Preferably, the surface of the control 42 which coincides with the front surface 23 is the front surface of the control 42, which is visible on the side of the front surface 23 as illustrated in FIG. 5. If the fastening part is not properly mounted, the operator can know it by noticing a step between the control and the front surface of the head when touching the front surface. Preferably, the control 42 is in abutment in the mounting position.

With this movement, once the control 42 is in abutment, the operator visually ensures that the fastening part is properly mounted.

In one aspect, the head 20 has an imprint 24 for receiving a tool. Advantageously, the imprint 24 is located on the front surface 23 of the head 20, preferably at the centre of the head 20, as illustrated in FIGS. 4 and 5. Just as example, the imprint is a TORX T30 type one, to receive a corresponding tool, for mounting and/or dismounting the fastening part 100.

Advantageously, once the fastening part 100 is unscrewed, the fastening part 100 could be clipped again.

Preferably, the fastening part is made of a plastic material. The fastening part 100 according to the disclosure may be manufactured by moulding a known plastic material, for example by thermoplastic injection, more particularly, technical plastics such as polyamides, acetal resins, thermoplastic polyesters. Advantageously, the fastening part 100 is made of known materials allowing withstanding ageing and temperature, and at the same time enabling the flexible tab 40 to deform and, preferably, the deformation of the flexible tab 40 is elastic.

In one aspect, the shank 60 includes additional fins made of metal, so-called claws, protrude from the surface of the shank 60 and extend towards the head 20. The additional fins made of metal may be integrated into the shank 60 according to known techniques, for example by overmoulding, mechanical assembly or ultrasonic welding.

FIG. 6 shows a side view of a fastening part 100 according to another aspect of the present disclosure. The fastening part 100 includes a body which comprises a head 20 and a shank 60. The head 20 has a rear surface 21 intended to be positioned opposite the first part 200. The shank 60 extends from the rear surface 21 of the head 20 and is intended to be inserted into the hole(s) of the parts to be assembled.

Preferably, the head 20 is round shaped. Preferably, the shank 60 is cylindrical shaped.

The shank 60 includes hooking fins 62 which project from the shank 60. At least one hooking fin 62 includes, at its free end, a double-head 63 serving as a thread.

The term “hooking fin” used in this description is technically interchangeable with the term “fin”.

The portion of the hooking fins 62 having a double-head 63 may be defined according to the need. In a preferred aspect, each hooking fin 62 is provided with a double-head 63.

Advantageously, the presence of a double-head at the end of a fin allows having a better clipping blade end stiffness.

Preferably and optionally, the head 20 and the flexible tabs can replicate the same features described hereinabove and illustrated in FIGS. 1 to 5, without being repeated herein.

In general, the fins 62 are fir tree like shaped. The fir tree like shaped fins 62 allow guaranteeing a solid fastening. The conventional fir tree like shaped fins 62 will hold the fastening part 100 in a hole. For example, the fastening part 100 with the fir tree like shaped fins 62 may be used to fasten a plastic deflector in a threaded hole.

The advantages and the technical effects of having double-headed fins will be detailed later on.

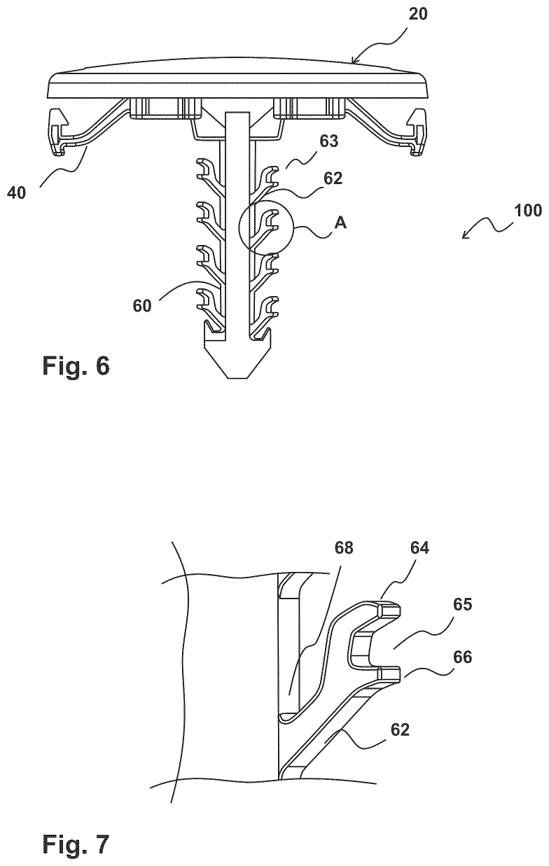

FIG. 7 shows a detail view of the area A in FIG. 6.

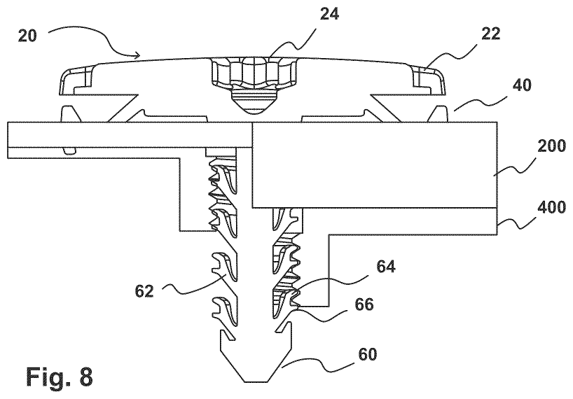

It is observed that the double-head 63 includes an upper head 64 and a lower head 66 separated by an intermediate space 65, these two heads 64, 66 defining a thread pitch. This will be explained later on with FIG. 8.

Preferably, at least one hooking fin 62 includes a thin area 68 between the shank 60 and the double-head 63. By “thin area”, it should be understood a reduced width compared to the double-head 63.

Having a double-headed end 63 makes the length of the thin area 68 shorter than a fin without double-head for example.

Advantageously, the compression and deformation until rupture of the fins is delayed, this thus allowing for a more robust hooking when the rivet 100 is pulled off.

FIG. 8 shows a sectional view of a fastening part according to one aspect, the rivet being received in a threaded hole. It is observed that the rivet 100 is received in the threaded hole, with the upper 64 and lower 66 heads in the helical grooves of the threaded hole. The double-head allows having twice as many contact points between the rivet 100 and the hole of the parts to be fastened. Advantageously, this allows creating a double hooking in the thread. This double hooking is particularly advantageous and offers in particular a better pull-off resistance.

In one aspect and more particularly, the lower head 66 has a predominant role in the pull-off resistance of the rivet 100. In parallel, the upper head 64 provides additional holding to reinforce this hold.

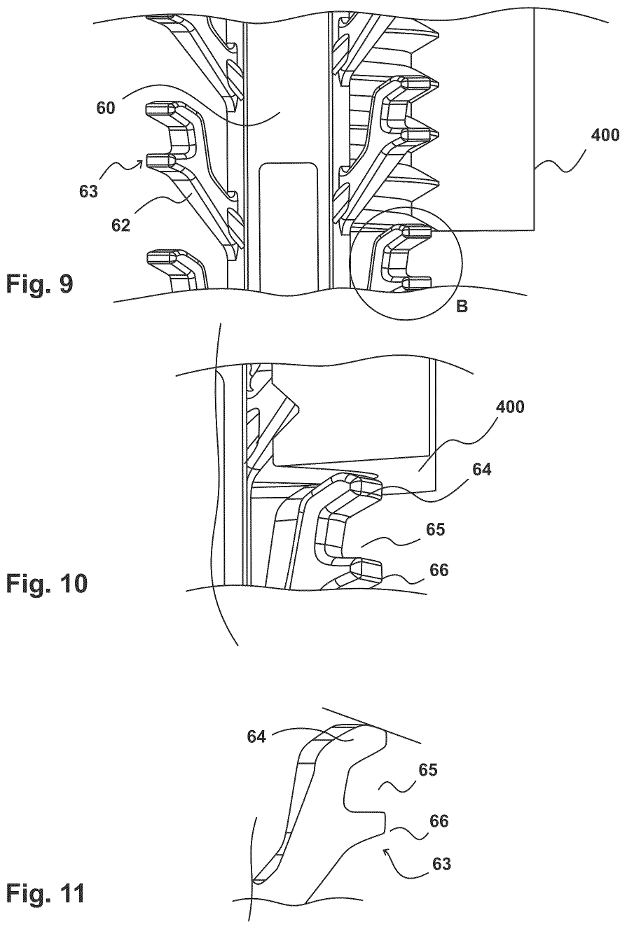

FIG. 9 shows a similar view of a fastening part according to one aspect, the rivet being received in a threaded hole. FIG. 10 shows a detail view of the area B in FIG. 9;

Preferably, the upper head 64 has a shape enabling unscrewing or simple pulling of the rivet 100. For example, the upper head 64 is provided with an inclined upper surface which accompanies the lead-in chamfer of the nut.

This also allows having a better unscrewing possibility.

This is particularly advantageous for through holes. In fact, with a through hole, the fins of the double-headed fir tree might pose a problem when they protrude from the nut opposite thereto, i.e., when the fins of the screw protrude from the other side of the nut. In this situation, a simple pull could cause a deformation of the fins, which would make re-insertion thereof into the nut difficult.

Advantageously, the upper portion of the upper head 64 has a specific geometry which allows a correct insertion while maintaining enough force for the fins to remain engaged in the thread, thereby serving as a screw. This means that the double-head allows an easy interlocking with clipping while providing a good pull-off resistance.

In a solution without a double-head, if these fins do not require more mounting force than these fins could generate in the thread (holding force), they simply risk moving inside the net, coming out and finally rotating in the void.

FIG. 11 shows a sectional view of the double-headed portion of a fin according to one aspect.

In one aspect, the upper head 64 is provided with an upper surface having a slope, as illustrated in FIG. 11.

The upper surface of the fin has a downward inclination whereas the fin itself is directed towards the head 20 of the rivet 100.

When observing the upper surface of the fin, one could observe a slope or an inclination which tilts downwards, i.e. it progressively descends towards the end of the upper head 64.

Other suitable geometries of the upper surface of the upper head 64 could also be considered.

### LIST OF REFERENCE SIGNS

TABLES 1

| References | Designations |
|---|---|
| 100 | Fastening part |
| 20 | Head |
| 21 | Rear surface |
| 22 | Notch |
| 23 | Front surface |
| 24 | Imprint |
| 26 | Setback |
| 40 | Flexible tab |
| 42 | Control |
| 44 | Arm |
| 60 | Stem |
| 62 | Fin; Hooking fin |
| 63 | Double-head |
| 64 | Upper head |
| 65 | Intermediate space |
| 66 | Lower head |
| 68 | Fin thin area |
| 200 | First part |
| 220 | First hole |
| 400 | Second part |
| 420 | Second hole |

What is claimed is:

1. A fastening part for fastening a first part to a second part having a hole, the fastening part includes a body which comprises:
a head having a rear surface intended to be positioned opposite the first part, and
a shank extending from the rear surface of the head and intended to be inserted into the hole;
characterised in that the shank includes fins which project from the shank, and that at least one fin includes, at its free end, a double-head serving as a thread.

2. The fastening part according to claim 1, wherein said at least one fin includes a thin area between the shank and the double-head.

3. The fastening part according to claim 1, wherein the double-head includes an upper head and a lower head separated by an intermediate space, these two heads defining a thread pitch.

4. The fastening part according to claim 1, wherein the fins are fir tree like shaped.

5. The fastening part according to claim 1,
characterised in that it includes at least one flexible tab which comprises an elastic arm one end of which is secured to the body of the fastening part, the arm including a control;
and in that the arm deforms so that the control fits in a predefined position with respect to the head, when the fastening part is in a mounting position in which the first part is fastened to the second part by the fastening part.

6. The fastening part according to claim 1, wherein the head includes at least one notch, corresponding to the predefined position, intended to receive the control in the mounting position of the fastening part.

7. The fastening part according to claim 1, wherein the head includes a front surface opposite to the rear surface, and a surface of the control, in the mounting position of the fastening part, is coincident with the front surface of the head.

8. The fastening part according to claim 1, wherein the head includes at least one setback, intended to accommodate the control once the control fits in the predefined position, and to prevent the control from moving during unscrewing.

9. The fastening part according to claim 1, wherein the arm of the flexible tab is secured to the rear surface of the head.

10. The fastening part according to claim 1, wherein the flexible tab is positioned on the side of the rear surface of the head.

11. The fastening part according to claim 1, wherein the arm of the flexible tab extends toward the edge of the head.

12. The fastening part according to claim 1, including at least one pair of flexible tabs, wherein the two flexible tabs of the pair of flexible tabs extend in two opposite directions.

13. The fastening part according to claim 11, including at least one pair of notches, each intended to receive one of the controls in the mounting position of the fastening part.

14. The fastening part according to claim 1, wherein the head has an imprint for receiving a tool.

15. The fastening part according to claim 1, being manufactured essentially of a plastic material.

16. The fastening part according to claim 1, wherein the shank includes additional fins made of metal, so-called claws, project from the surface of the shank and extend towards the head.
